(19) United States
(12) Patent Application Publication
(10) Pub. No.: US 2013/0222767 A1
(43) Pub. Date: Aug. 29, 2013

(54) METHODS AND SYSTEMS FOR DETECTING PERIPAPILLARY ATROPHY

(71) Applicants: Jun CHENG, Singapore (SG); Jiang LIU, Singapore (SG); Wing Kee Damon WONG, Singapore (SG); Ngan Meng TAN, Singapore (SG); Seang Mei SAW, Singapore (SG); Tien Yin WONG, Singapore (SG)

(72) Inventors: Jun CHENG, Singapore (SG); Jiang LIU, Singapore (SG); Wing Kee Damon WONG, Singapore (SG); Ngan Meng TAN, Singapore (SG); Seang Mei SAW, Singapore (SG); Tien Yin WONG, Singapore (SG)

(21) Appl. No.: 13/651,309

(22) Filed: Oct. 12, 2012

(30) Foreign Application Priority Data
Oct. 12, 2011 (SG) 201107459-8

(51) Int. Cl. A61B 3/14

(52) U.S. Cl. CPC A61B 3/14
USPC 351/246

(57) ABSTRACT

A method is presented for deciding whether an eye exhibits peripapillary atrophy (PPA). It includes a preliminary step of extracting from an image of the eye a region-of-interest which would be affected if the eye exhibits peripapillary atrophy, which is a region which surrounds the optic disc, and then processing the region in a way which mimics the processing of the cortex, to derive a plurality of numerical measures (biologically-inspired features, BIF). A decision step is then performed using the BIF, for example using an adaptive system which has been subject to a supervised learning process. Preferably, the region-of-interest is partitioned into a plurality of sub-regions, and the BIF are derived as a corresponding plurality of numerical measures for each of the sub-regions. The BIF preferably include intensity units which take values indicative of centre-surround intensity difference; and colour units which take values indicative of centre-surround difference in a parameter characterizing colour in the image. Further, the BIF preferably include direction-specific units.

### FIELD OF THE INVENTION

The present invention relates to methods and systems for detecting a characteristic of an eye, such as that the eye exhibits peripapillary atrophy (PPA). The result of the detection may be used as an indication of a propensity to myopia.

### BACKGROUND OF THE INVENTION

In the field of medical imaging, an expert, usually a radiologist, would perform diagnosis of a disease by observing and interpreting acquired medical images. Such a trained professional would usually be able to ascertain a condition on the basis of the presence of abnormal visual symptoms in the images. These visual symptoms are perceptual physiological indicators used as medical evidence for diagnosis of many diseases. Some examples of the perceptual physiological indicators commonly identified are: peripapillary atrophy in a myopia eye (that is, an eye suffering from myopia); drusen in age related macular degeneration, trabecular meshwork in glaucoma, etc. In these applications, doctors/ophthalmologists examine a particular region to determine the presence/absence and/or high/low probability of a particular disease based on the visual appearance of the region. Very often, these perceptual physiological indicators are examined manually. However, it is often time consuming, subjective and expensive. Automatic detection of these indicators is thus beneficial to save workload and facilitate large-scale clinical use.

As noted above, one example of an indicator is peripapillary atrophy (PPA), which means the atrophy of pre-existing tissue. Because of its association with some eye diseases, it is an important indicator in eye disease diagnosis, and thus it is important to evaluate the presence of PPA from retinal images. Peripapillary atrophy (PPA) refers to a white or pigmented crescent-shaped area adjacent to the optic disc. As its name suggests, PPA is a recession of the tissue. It is associated with some eye diseases such as myopia [1]. Myopia is a major public health problem, with enormous and often under-estimated economic and medical costs, and the early detection and management of degenerative eye diseases is of utmost importance in the care of myopic adults. PPA is an early sign of myopia. In myopia eyes, PPA is found to be highly correlated with myopia: the more myopic the eye, the larger the PPA region [1]. Thus, it is important to determine the presence/absence of PPA.

FIGS. 1(a)-1(d) show four sample retinal images. FIGS. 1(e)-1(h) show images that are obtained by expanding the images of FIGS. 1(a)-1(d), respectively, and concentrating on the portions of the images surrounding the optic disc. PPA is present in the images shown in FIGS. 1(e)-1(g), but absent in the image shown in FIG. 1(h). Experienced ophthalmologists are able to determine the presence of FPA from such images. However, it is time consuming and subjective to examine many images, especially in a screening program. To save workload and facilitate large-scale clinical use, it is essential to have a precise, efficient and cost effective system to determine the presence of PPA automatically.

A disc difference approach is proposed in [2]. A variational level set is used to determine candidate regions for PPA analysis. This approach is broadly successful, but has two limitations: first, it relies on the PPA region having a strong boundary, so that the level set does not cross over the PPA region. However, such a boundary is not present in some cases, such as the image of FIG. 1(f). Second, the level set based optic disc segmentation used in the algorithm often incorrectly identifies PPA as part of optic disc especially when the PPA region is relatively small. The error in optic disc segmentation propagates to the feature extraction and thus the final result.

The “PAMELA” technique described in [3, 12] combines texture features with the disc difference method of [2] and overcomes the first limitation by extracting features from the four quadrants of the region around the segmented optic disc. The texture features are calculated based on an entropy function. However, global entropy features from the four quadrants are often much less significant for PPA with mild size. The system has only been tested in a small dataset which consists of 40 normal cases and 40 cases with very large PPA. It has not been tested on screening data.

### SUMMARY OF THE INVENTION

The present invention aims to provide methods and systems for the detection of whether an eye exhibits a characteristic such as PPA.

It is motivated by the observation that humans demonstrate the ability to capture instantly the ‘gist’ of an image despite its size, while computers cannot perform as well as humans on visual recognition. Based on this observation, biologically inspired features (BIF) [4][5][6] have been used in computer vision systems to perform tasks such as scene classification and gait recognition which are similar to those which humans have natural ability to carry out. BIF are numerical parameters which are generating by processing an image in a way which mimics the process of cortex for visual perception. The invention is based on the realisation that the recognition of PPA in a fundus image, although it is not one of the tasks which humans perform in everyday life, has some subtle similarities to those tasks.

In particular, since medical professionals can train themselves to recognise the presence of perceptual physiological indicators such as PPA from the visual information, the invention proposes using BIF for automatic detection of perceptual physiological indicators. However, in contrast to conventional scene classification tasks in which the scene varies greatly from one image to another, the ‘scene’ here differs slightly only in a small area around optic disc. When medical professionals examine the images, they focus on a specific region.

Thus, the invention proposes in general terms that a method of deciding whether an eye exhibits a certain characteristic (such as PPA) is performed using an image of the eye. The image is defined by intensity values at each of a set of pixels (there are typically multiple intensity values for each pixel, corresponding to different colours). A preliminary step of the method is to extract from the image a region-of-interest which would be affected if the eye exhibits that characteristic (e.g. in the case of PPA, this region is a region which surrounds the optic disc), and then processing the region in a way which mimics the processing of the cortex, to derive a plurality of numerical measures (BIF). The numerical measures are then used to decide whether the characteristic is present. All the steps of the method are preferably performed automatically. The decision step may be accomplished using an adaptive system which has been subject to a supervised learning process.

Preferably, the region-of-interest is partitioned into a plurality of sub-regions, and the BIF are derived as a corresponding plurality of numerical measures for each of the sub-regions. For example, each BIF can be obtained in the form of a function spanning the region-of-interest, and this function is used to obtain an average value of the function over each sub-region.

The BIF functions are preferably each produced by processing the region using filters which filter the image at a plurality of distance scales, and combining the results. This is because, as described in detail below, many “units” of the visual processing portion of the cortex perform a processing operation which can be modelled by combining outputs of functions generated on different distance scales (that is, levels of resolution). Each of the BIF functions may be generated by performing an operation which models the function of a corresponding one of the units.

Some of the BIF are computed by a set of “centre-surround” operations akin to the visual receptive fields. Typical vision neurons are most sensitive in a small region of the visual space (the centre), while stimuli presented in a broader, weaker antagonistic region concentric with the centre (the surround) inhibit the neuronal response. BIF features which mimic “centre-surround” operations are particularly well-suited to detecting locations which locally stand out from their surround. Centre-surround is implemented in preferred embodiments of the invention as the difference between fine and coarse scales.

Specifically, the operations used to generate the BIF preferably include operations to model:
intensity units which take values indicative of centre-surround intensity difference; and/or
colour units which take values indicative of centre-surround difference in a parameter characterizing colour in the image.

Further, the BIF preferably include direction-specific units. Each direction-specific unit corresponds to a single direction in the image. A direction-specific unit for a given sub-region indicates a directionality of that sub-region of the image in the corresponding direction. It is generated using filters which perform a filtering operation to emphasize components (such as edges) having extension in the corresponding direction. The filters may be Gabor filters.

A given direction-specific unit may be produced using a C1 pooling operation, which combines the results obtained for a plurality of distance scales. In other words, for each direction, the filtering may performed successively using parameters which produce different distance scales in the Gabor filters for that direction. Following this filtering, the results at neighbouring distance scales are combined pairwise, to produce a plurality of direction-specific units for that direction.

Preferably, dimensionality reduction is performed to reduce the complexity of the computational task which the adaptive system has to perform. This is motivated by the observation that a BIF can be considered as a low dimensional feature artificially embedded in a high dimensional space.

In the case that the characteristic is PPA, the region-of-interest is obtained by deriving an estimate of the position of the optic disc, and defining the region-of-interest as a ring having the optic disc as an inner boundary. The region-of-interest is preferably mapped to a two dimensional rectangular area, and the sub-regions are defined as rectangular regions within the rectangular area.

The step of estimating the position of the optic disc may include a thresholding step based on an intensity of the image at respective pixels, obtaining the boundary of the largest connected group of pixels having an intensity above the threshold, and smoothing the boundary to detect obtain an ellipse.

The thresholding step may be performed using a threshold which is computed adaptively for each image based on prior knowledge of typically optic discs, such as in the form of parameters indicating the maximum and minimum numbers of pixels commonly present in an optic disc.

The smoothing step may be performed using elliptical Hough transforms.

The invention may be used to perform PPA detection while reducing the workload of medical professionals. For example it can be used as a low-cost screening method for identifying individuals for whom further screening should be employed, as a preliminary to a step of intervention to treat myopia or reduce its likelihood of occurrence.

The invention may be expressed as a method. It may alternatively be expressed as a computer system for carrying out the process described above, or a computer program for performing the method (for example stored in non-transitory form on a tangible recording medium, such as an optical disk).

The term “automatic” is used in this document to refer to a process carried out by a computer system substantially without human involvement (except, optionally, for initiation). This is in contract to the term “semi-automatic”, which refers to a process which is carried out with human intervention (for example manually indicating a region-of-interest, or specifying a parameter) during the process. The term “computer system” refers to a computer of any type, including a PC, server or mobile device, having a processor and a tangible data storage device for storing non-transitory program instructions for performance by the processor to cause the processor to carry out a method. The computer further has a display device for displaying results of the process, and/or a data interface for transmitting the results of the process outside the computer system.

### BRIEF DESCRIPTION OF THE FIGURES

An embodiment of the invention will now be described for the sake of example only with reference to the following figures, in which:

FIGS. 1(a)-1(d) show fundus images;

FIGS. 1(e)-1(h) show corresponding enlarged portions of the images shown in FIGS. 1(a)-1(d), including the optic disc;

FIG. 2 is a flow diagram showing the steps of a method which is an embodiment of the present invention;

FIG. 3 shows sub-steps of one of the steps of FIG. 2;

FIGS. 4(a)-4(g) show how a single fundus image is processed during the first two steps of FIG. 2; and

FIGS. 5(a)-5(d) show schematically how an image is transformed during the first three steps of FIG. 2.

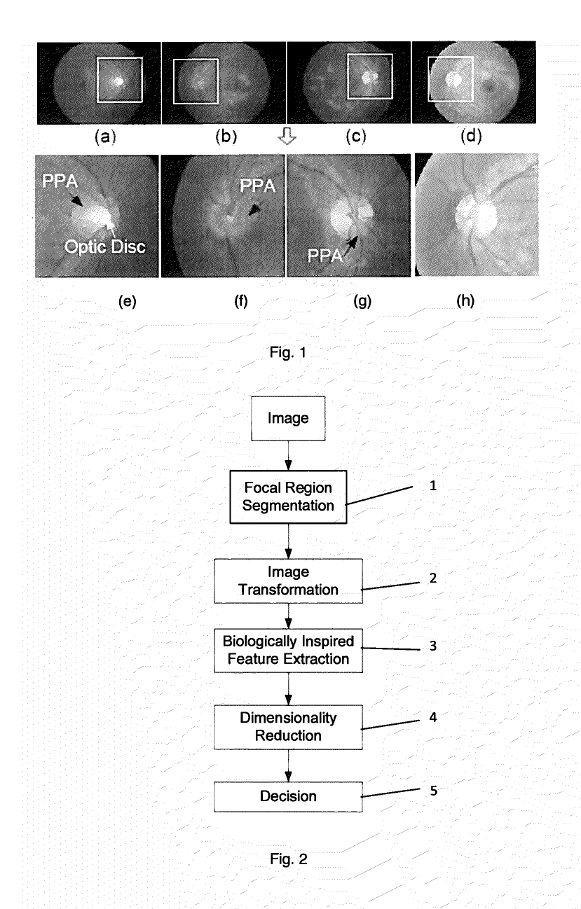

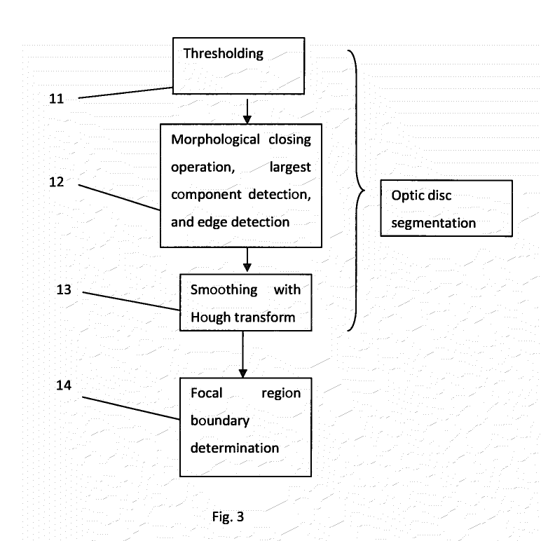

### DETAILED DESCRIPTION OF THE EMBODIMENT

Referring to FIG. 2, a flowchart is shown of an embodiment of the invention. The input to the flowchart is a single image of the rear of an eye: typically a conventional fundus image. The embodiment is one for determining whether the eye exhibits PPA.

In a first step 1, a “focal region” is identified in the image. This focal region is a region which includes the periphery of the optic disc, and thus includes the area which typically includes PPA.

In step 2, the focal region is transformed into a rectangular transformed image. A plurality of sub-regions are defined, such as by partitioning the focal region into non-overlapping rectangular blocks which collectively cover the focal region.

In step 3, a plurality of biologically-inspired features (BIF) are obtained for each of the sub-regions.

In step 4, the BIF for all of the sub-regions are subject to an optional dimensionality reduction step, which produces a plurality of second numerical measures. The number of second numerical measures is less than the number of BIF extracted in step 3.

In step 5, the second numerical measures are used by an adaptive system to generate at least one value indicative of a decision of whether PPA is present in the eye.

These steps are now described in detail:

### Step 1: Focal Region Segmentation

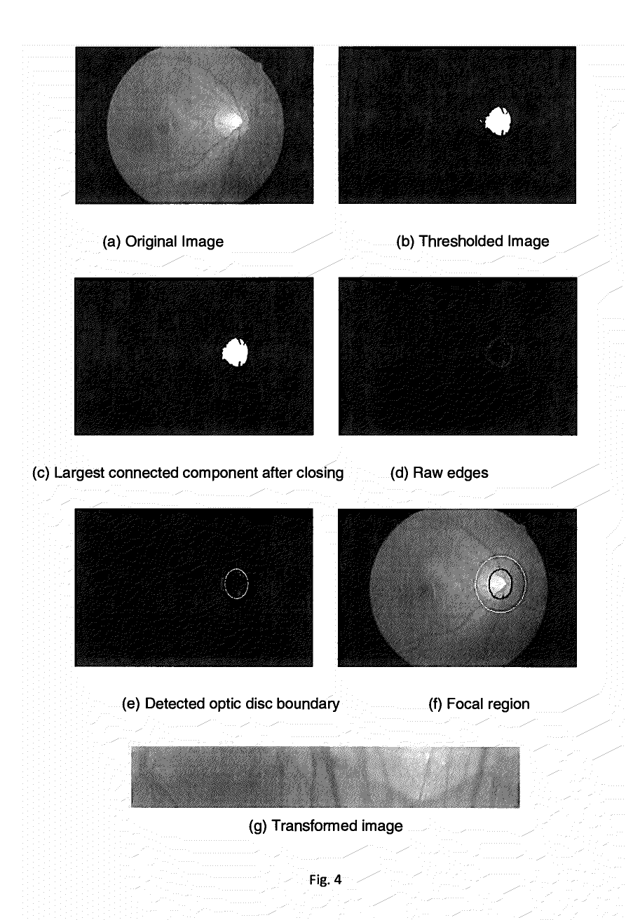

The motivation for performing this step is that extracting the BIF from the whole image would be computational expensive and unnecessary. The region-of-interest varies in different embodiments of the invention, depending on which characteristic of the eye each embodiment is intended to detect. In the present embodiment, which performs PPA detection, the region-of-interest is that around the optic disc, as this is the area in which PPA might appear. Thus, the embodiment employs “focal BIF”. Focal BIF refers to extracting and using biologically-inspired features (BIF) from a region-of-interest referred to as the “focal region”. The sub-steps 21-24 of step 1 are shown in FIG. 4.

To segment the focal region such that the perceptual physiological indicators are included in the region, it is important to find a boundary which can be detected with sufficient accuracy. Such a boundary is able to differentiate areas of the eye which exhibit significant perceptual physiological indicators from other areas which do not include such indicators. In this application, the focal region is the area outside the optic disc, and the boundary of the optic disc is used as the inner boundary of the focal region.

Thus, the first task of step 1 is to locate and obtain the boundary of the optic disc (“optic disc segmentation”, which is sub-steps 11-13 of FIG. 4). Optic disc localization algorithms as in [9] [10] [11] can be used. In this embodiment, the fringe is first trimmed away from the retinal image as in [11] at the beginning of step 11, and then the area with the brightest spot is located. Various other algorithms have been proposed for optic disc segmentation including the level-set approach [2], active contour approach [8], etc. However, these approaches are sensitive to initialization. Moreover, they do not perform well in images with PPA and often mistake PPA for the optic disc. Consequently, the errors propagate to PPA detection. As the main purpose here is to detect PPA, good optic disc segmentations would make PPA remain in the focal region.

Sub-step 11 is a thresholding sub-step, in which the embodiment estimates the optic disc via threshold as follows.

First, the embodiment identifies pixels in the image brighter than a threshold T as the candidate brightest spot. As suggested in [2], the embodiment applies this threshold only to the red channel of the image since this channel provides a better result than the other channels as it avoids the most gradients of blood vessels. Mathematically, we have:

$$E ( x , y ) = \{ \begin{matrix}1 & if {I}_{r} ( x , y ) > T \\ 0 & otherwise\end{matrix} \qquad ( 1 )$$

where Iᵣ is the value of red channel at a location (x,y) in the fundus image.

The threshold T is determined as follows. Since different fundus images may have different respective levels of illumination, we determine the threshold T adaptively for each image. This is done in the following. First, we derive an average intensity of the image Īᵣ. Second, T is set to initialized to a value Īᵣ+ΔT₀, where the value of ΔT₀ is set empirically (that is, by trial-and-error).

Thirdly, T is adaptively reduced or increased to a new value Īᵣ+ΔT if the number of pixels greater than T is not within the range of typical optic disc size, i.e.,

$$Δ T = \{ \begin{matrix}Δ {T}_{0} , & if {D}_{min} \leq f ( {I}_{r} , {\overline{I}}_{r} + Δ {T}_{0} ) \leq {D}_{max} \\ {arg max}_{Δ T} f ( {I}_{r} , T ) \geq {D}_{min} , & if f ( {I}_{r} , {\overline{I}}_{r} + Δ {T}_{0} ) < {D}_{min} \\ {arg max}_{Δ T} f ( {I}_{r} , T ) \leq {D}_{max} , & if f ( {I}_{r} , {\overline{I}}_{r} + Δ {T}_{0} ) > {D}_{max}\end{matrix} \qquad ( 2 )$$

where f(I,T) denotes a function to compute the number of pixels in image Iᵣ with pixel values greater than T. Dₘᵢₙ and Dₘₐₓ are two empirically determined threshold values which respectively represent the minimum and maximum number of pixels in a typical optic disc. The following steps are used implement Eqn. (2):

1) Let T=Īᵣ+ΔT₀ and compute f(Iᵣ,T)

2) If f(Iᵣ,T)<Dₘᵢₙ, decrease T stepwise until f(Iᵣ,T)≧Dₙᵢₙ.

3) If f(Iᵣ,T)>Dₘₐₓ, increase T stepwise until f (Iᵣ,T)≦Dₘₐₓ.

In sub-step 12, a morphological closing process is applied to remove noise. Then the largest object, i.e., the largest connected group of pixels, having an intensity above T is obtained The edge of the largest group of pixels is then obtained. This is a first estimate of the optic disc. This completes sub-step 12.

It has been found that the boundary of the optic disc obtained in this way is often irregular and unsuitable to extract BIF from it. Thus, in sub-step 13 the embodiment smoothes the boundary to determine better estimate of the boundary of the optic disc. As the optic disc is approximately an ellipse, we apply constrained elliptical Hough transform to get the disc boundary. Note that an ellipse can denoted by the position (x,y) of its centre, parameters (a,b) denoting its semi-major and semi-minor axes respectively, and an angle co denoting the direction in which the major axis extends. Step 13 is carried out as follows:

1) Set trial parameters (a, b, φ) for the ellipse by selecting respective values for each of the parameters within pre-determined respective ranges. These pre-determined ranges are chosen empirically but so as to include the typical values for a and b which are consistent with the typical size of an optic disc. Together the ranges define a parameter space.
2) An accumulator A is defined for each point in a 5-dimensional space which includes not just the dimensions (x,y) but also dimensions (a, b, φ). Initially A is set to zero for each point in the 5-D space. For each point (xe, ye) on the boundary obtained in step 12, we draw an auxiliary ellipse centred at (xe, ye) and with parameters with (a, b, φ). For each point on the boundary of the auxiliary ellipse, we increment A by one at the location in the 5-D space which corresponds to that point and to (a, b, φ).
3) Update (a, b, φ) and repeat step 2) for all (a, b, φ) from the parameter space.
4) Find the maximum value in A to get an ellipse centered at (x1, y1) and corresponding parameters (a1, b1, φ1).
5) If b1/a1>γ, then find the maximum value in A subject to b/a≦γ to get a second ellipse centered at (x2, y2).
6) Determine the optic disc boundary as the second ellipse if A(x2, y2)/A(x1, y1)>TA where TA is an empirically determined value. Otherwise, determine the optic disc boundary as the first ellipse. In either case, let us call the optic disc boundary the ellipse having centre (\hat{x},ŷ) and parameters (â,\hat{b},\hat{φ}).

The idea is that if b1/a1>γ, there is a higher chance that the first detected ellipse include some of PPA. Thus we would further check if there is another slightly weaker ellipse with b/a≦γ. If so, the second one is more likely to be the disc boundary. This reduces the risk of mistakenly considering some of the PPA to be part of the optic disc.

After obtaining the ellipse with parameters (â, \hat{b}, \hat{φ}) at (\hat{x},ŷ), in sub-step 4 the focal region is defined using this ellipse and a larger ellipse (â+r, \hat{b}+r, \hat{φ}) at same center (\hat{x}, ŷ). Specifically, the region of the image between the two ellipses is defined as the focal region, and is an elliptical ring.

### Step 2: Focal Region Transformation

In step 2 of the flow of FIG. 2, the focal region image is transformed from an elliptical ring to a rectangular area. In other words, a mapping is defined between the elliptical ring and the rectangular area. This mapping is based on a “pivot point” which is the centre (\hat{x}, ŷ). Each point in the elliptical ring has a certain angular position about the pivot point, and a certain distance from the pivot point. Under the mapping, the x-coordinate of the corresponding location in the rectangular area is proportional to the angular position. The y-coordinate of the corresponding location in the rectangular area is proportional to the distance to that point from the pivot point minus the distance from the pivot point to the point on the inner ellipse at the same angular position.

Rectangular sub-regions of the rectangular area are now defined. They are non-overlapping, and collectively span the rectangular area.

FIG. 4 illustrates an example of steps 1 and 2. FIG. 4(a) is the original image. FIG. 4(b) is the output after comparing the red channel in FIG. 4(a) with the adaptively determined threshold, i.e. the object obtained in sub-step 11. The largest object is shown in FIG. 4(c), with its boundary shown in FIG. 4(d), i.e. the first estimate of the optic disc obtained at the end of sub-step 12. From the edges in FIG. 4(d), ellipse fitting through elliptical Hough transform is used to detect the boundary of the optic disc, as shown in FIG. 4(e), which is obtained in sub-step 13. In sub-step 14, the focal region is obtained as a ring area as shown in FIG. 4(f), where the inner ellipse is the detected ellipse. Finally, in step 2 the ring region of FIG. 4(f) is transformed to a rectangular area as in FIG. 4(g).

### Step 3 Extraction of the Biologically Inspired Features (BIF)

The BIF in the embodiment are produced using 34 feature maps. Each feature map is defined spanning the region-of-interest. Each map is produced by a process mimicking the process carried out by a corresponding type of visual processing “unit” within a human cortex. The feature maps consist of 6 feature maps from (i.e. modelling the function of) intensity units, 12 feature maps from color units, and 16 feature maps from direction-specific C1 units.

The feature maps modelling intensity units are obtained by convolving dyadic Gaussian pyramids with the intensity channel of the colour fundus image. The features correspond to the neurons of mammals which are sensitive to dark centers on bright surrounds or vice versa [7]. Nine spatial scales are generated with a ratio from 1:1 (level 0) to 1:256 (level 8). The intensity feature maps are obtained by the center-surround difference operation between center levels c=2, 3, 4 and surround levels s=c+d, with d=3, 4. Thus, six feature maps are computed at (c,s) levels of (2,5), (2,6), (3,6), (3.7). (4,7), and (4,8). Because of the scale difference, maps of surround levels are interpolated to be the same size as the corresponding centre levels, and then they are subtracted to generate the relevant feature maps, i.e., I(c,s)=|I(c)−Interp_{s-c}(I(s))|, where Interp_{s-c}( ) denotes the interpolation from level s to level c.

The colour units are inspired by the ‘colour double-opponent’ system in the cortex [7]. Neurons are excited by a colour (e.g., blue) and inhibited by another colour (e.g., yellow) in the center of receptive field, so are neurons in the surround. Herein, four colour channels are used: R=r−(g+b)/2. G=g−(r+b)/2, B=b−(r+g)/2 and Y=r+g−2(|r−g|+b). For each colour channel (A, G, B, and Y), dyadic Gaussian pyramids are used to generate nine spatial scales, similar to the scales used to generate the intensity units. Two color pairs R-G and B-Y are used. The feature maps are computed as the across scales center-surrounding differences. Similarly to the computation of intensity units, surround maps are interpolated to be the same size as the corresponding center maps and their difference is computed as: RG(c, s)=|(R(c)−G(c))−Interp_{s-c}(R(s)−G(s))| and BY (c,s)=|(B(c)−Y(c))−Interp_{s-c}(B(s)−Y(s))|.

The direction-specific C1 units pool the outputs of multiple S1 units, which correspond to simple cells in the S1 layer of the visual cortex. Gabor functions are used as filters (“Gabor filters”) for feature extraction due to their similarity to the receptive field profiles in simple cells in the S1 cortical layer. The Gabor functions are all self-similar, and are generated from a single “mother function” which can be written as:

F(x,y)=exp(−(x₀²+y²y₀²)/(2δ²))×cos(2πx₀/λ),

where δ and λ are predetermined parameters and x₀=x cos θ+y sin θ, y₀=−x sin θ+y cos θ. Each Gabor filter corresponds to specific values of the parameters x, y and θ. The values of x and y decide the scales of the corresponding Gabor filter, and θ controls its orientation. In this embodiment, eight scales with a range of sizes from 7×7 to 21×21 pixels with a step of two pixels are used. For example, for the smallest scale x and y may both be chosen to be equal to 7. Four orientations are considered: θ=0°, 45°, 90°, and 135°. Thus, a total of 32 feature maps are obtained in S1 units. The direction-specific C1 units are obtained by pooling the S1 units with the same value of 0 pairwise (i.e. each direction-specific C1 unit is produced by pooling a pair of S1 units with adjacent scales and with an identical orientation). “Pooling” refers to taking the maximum of two values. The pooling is carried out at each point in the space.

Thus, for each orientation, we obtain four direction-specific C1 units by pooling respectively the S1 units with the 7-pixel and 9-pixel scales; the 11-pixel and 13-pixel scales; the 15-pixel and 17-pixel scales; and the 19- and 21-pixel scales. Thus, 16 feature maps are obtained in the form of direction-specific C1 units.

Thus, in summary, a color image is represented by 34 feature maps, wherein 16 feature maps are obtained from C1 units, 6 intensity feature maps are obtained from 6 intensity filters convoluted with the input image, and 12 color feature maps are obtained from 12 color filters convoluted with the input image.

Each feature map is divided into m×n non-overlapping subregions, corresponding to the m×n sub-regions of the region-of-interest. Each of the 34 features maps is then used to obtain a respective numerical value for each of the m×n non-overlapping regions. This numerical value is the mean value of the corresponding feature map over the corresponding sub-region. Since each image is represented by 34 feature maps and each feature map is decomposed into m×n sub-regions, we have a total of 34 nm mean values as the BIF features for each image.

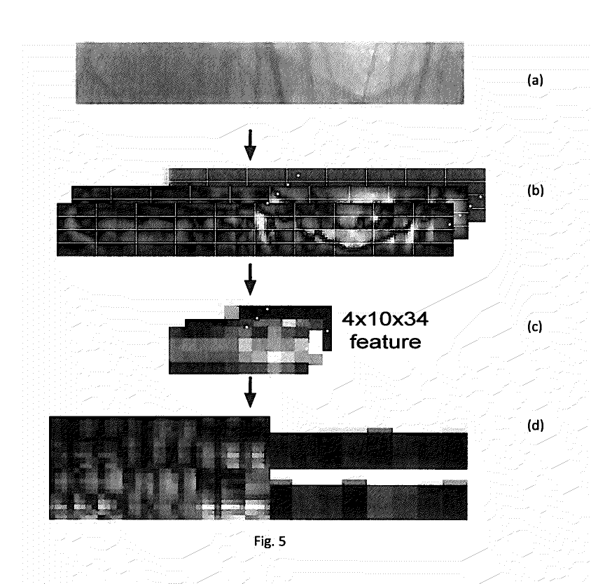

FIG. 5 shows schematically an example with feature maps divided into 4×10 sub-regions. FIG. 5(a) shows the region-of-interest. FIG. 5(b) shows how it is divided into 4×10 sub-regions. FIG. 5(c) shows schematically converting this into 4×10×34 features. In FIG. 5(d) these 4×10×34 (that is, 1360) features are represented as a two-dimensional area including 1360 squares, with each square having an intensity which is equal to a corresponding BIF for a corresponding one of the sub-regions.

### Step 4

Optionally, a step 4 of dimensionality reduction is performed, to reduce obtain a set of second numerical measures from the BF features obtained in step 3. The number of second numerical measures is less than the number of BF features obtained. The dimensionality reduction may be performed for example by principal component analysis (PCA).

### Step 5

Support vector machines (SVM) are commonly used adaptive systems (optimization tools) for solving machine learning problems, and an SVM was used as the adaptive system employed in the embodiment. The output of step 3, or of step 4 if step 4 is present, is passed to the SVM which has been trained by supervised learning to generate an output (for example 0 or 1) which is indicative of whether PPA is present in the image.

### Experimental Results

A total of 1584 images from Singapore cohort study of the risk factors for myopia (SCORM) retina study are used. The images are collected from 792 school children aged 8 to 11 years. Among the 792 children, 677 are with myopia and 115 are without myopia. All the subjects in the study had retinal photography performed by trained staff using the retinal camera. The presence of PPA has been manually determined and a total of 240 negative (without PPA) and 1344 positive (with PPA) samples. This included various sizes of PPA from mild to extensive

A total of 240 sample images including 120 positive and 120 negative samples are randomly drawn from the database for the training. The rest of images are used in the testing. For simplicity. PCA is used to reduce the dimensionality to 80 based on suggestions in [4].

The prior algorithms shown in [2, 3] are used for comparison in the testing. In the proposed BIF based approaches, the adaptive threshold is used for disc segmentation to determine the focal region. The region is transformed to rectangular and biologically inspired features are extracted from it. Support vector machines are used to train the classifiers for testing. Table 2 summarizes the sensitivity and specificity by the method. The results show that the embodiment performs significantly better than the method of [2, 3], and thus that a BIF-based approach may be better than the prior texture and/or disc difference based approaches.

TABLE 2
Performance comparison

| Method | The method of [2] | The method of [3, 12] | Embodiment |
|---|---|---|---|
| Specificity | 58.2% | 72.4% | 92.0% |
| Sensitivity | 62.4% | 75.2% | 92.8% |
| Overall | 60.3% | 73.8% | 92.4% |

In summary, the proposed BIF based approach can achieve accuracy more than 90% in detecting PRA. It can be used to save the workload of ophthalmologist. The technology can also be generalized for other visual cue detection in images from other imaging modality in other medical applications.

### REFERENCES

[1] S. M. Hyung, D. M. Kim, C. Hong, and D. H. Youn, “Optic disc of the myopic eye: Relationship between refractive errors and morphometric characteristics,” J. Ophthalmol., vol. 6, pp. 32-35, 1992.
[2] N. M. Tan, J. Liu, D. W. K. Wong, J. H. Lim, Z. Zhang, S. Lu, H. Li, S. M. Saw, and T. Y. Wong L. Tong, “Automatic detection of pathological myopia using variational level set,” Int. Conf. of IEEE Eng. in Med. and Bio. Soc., pp. 3609-3612. 2009.
[3] J. Liu, D. W. K. Wong, J. H. Lim, N. M. Tan, Z. Zhang, H. Li, F. Yin, B. H. Lee, S. M. Saw, L. Tong, and T. Y. Wong, “Detection of pathological myopia by pamela with texture-based features through an svm approach,” Journal of Healthcare Engineering, vol. 1, no. 1, pp. 1-11, 2010.
[4] C. Siagian and L. Itti, “Rapid biologically-inspired scene classification using features shared with visual attention,” IEEE Trans. Pattern Anal. Mach. Intell., vol. 31, no. 2, pp. 260-274. 2008.
[5] D. Song and D. Tao, “Biologically inspired feature manifold for scene classification,” IEEE Trans. Image Processing., vol. 19, no. 1, pp. 174-184, 2010.
[6] Y. Mu and D. Tao, “Biologically inspired feature manifold for gait recongition,” Neurocomputing., vol. 73, pp. 895-902, 2010.
[7] L. Itti, C. Koch, and E. Niebur, “A model of saliency-based visual attention for rapid scene analysis,” IEEE Trans. Pattern Anal. Mach. Intell., vol. 20, no. 11, pp. 1254-1259, 1998.
[8] Lowell, J., Hunter, A., Steel, D., Basu, A., Ryder, R., Fletcher, E., & Kennedy, L. (2004). Optic nerve head segmentation. IEEE Transactions on Medical Imaging, 23(2), 256-264. Retrieved from http://dx.doi.org/10.1109/TM!.2003.823261
[9] A. Hoover and M. Goldbaum, “Locating the optic nerve in a retinal image using the fuzzy convergence of the blood vessels,” IEEE Trans. on Medical Imaging, vol. 22, pp. 951-958, 2003.
[10] M. Foracchia, E. Grisan. and A. Ruggeri, “Detection of optic disc in retinal images by means of a geometrical model of vessel structure,” IEEE Trans. on Medical Imaging, vol. 23. no. 10, pp. 1189-1195, 2004.
[11] Z. Zhang, B. H. Lee, J. Liu, D. W. K. Wong, N. M. TAN, J. H. Lim, F. S. Yin, W. M. Huang, and H. Li, “Optic disc region-of-interest localization in fundus image for glaucoma detection in argali,” Proc. of Int. Conf. on Industrial Electronics & Applications, pp. 1686-1689, 2010.
[12] Liu Jiang; Wong Wing Kee Damon; Lim Joo Hwee; Tan Ngan Meng; Zhang Zhuo; Li Huigi; Lu Shijian; S. M. Saw, Louis Tong, Wong Tien Yin. Methods and Systems for Pathological Myopia Detection, PCT filed 2009.

What is claimed is:

1. A method of deciding whether an eye exhibits peripapillary atrophy (PPA), the method comprising:
(i) deriving a region-of-interest within an image of the eye, the image being defined by intensity values at a set of pixels;
(ii) using the region-of-interest to derive a plurality of numerical measures (BIF), by subjecting the region to respective processing operations which mimic corresponding cortical visual processing operations; and
(iii) using said numerical measures to decide whether the eye exhibits peripapillary atrophy.

2. A method according to claim 1 in which the region-of-interest corresponds to the portion of the eye surrounding the optic disc of the eye.

3. A method according to claim 2 in which the step (i) of deriving the region-of-interest includes deriving an estimate of the position of the optic disc, and defining the region-of-interest as a ring having the optic disc as an inner boundary.

4. A method according to claim 3 in which the estimate of the position of the optic disc is performed by:
a thresholding step based on an intensity of the image_{:}
obtaining the largest connected group of pixels having an intensity above the threshold,
obtaining the boundary of the largest connected group of pixels; and
smoothing the boundary to obtain an ellipse.

5. A method according to claim 4 in which the thresholding step is performed using a threshold which is obtained from the image, the threshold being obtained to give a number of pixels having an intensity above the image which is consistent with predefined values representing the maximum and minimum number of pixels commonly present in an image of an optic disc.

6. A method according to claim 4 in which the smoothing step is performed using elliptical Hough transforms.

7. A method according to claim 1, further including partitioning the region-of-interest into a plurality of sub-regions, step (ii) being performed to obtain a plurality of said numerical measures for each of said sub-regions.

8. A method according to claim 7 when dependent on claim 3, the method further including transforming the ring-shaped region-of-interest into a rectangular area, the sub-regions being defined as rectangular regions collectively spanning the rectangular area.

9. A method according to claim 1 in which in step (iii) an adaptive system generates a determination of whether the eye exhibits peripapillary atrophy.

10. A method according to claim 9 in which step (iii) comprises performing dimensionality reduction on said numerical measures, and inputting the result to the adaptive system.

11. A method according claim 1 in which the plurality of numerical measures are obtained using:
intensity feature maps obtained by convolving intensity filters with the region-of-interest;
colour feature maps obtained by convolving colour filters with the region-of-interest; and/or
direction-specific feature maps, each direction-specific features map being obtained by pooling the results of filtering the image using a plurality of direction-specific filters which filter the image in the same direction but at different distance scales.

12. A method according to claim 3, in which the plurality of numerical measures for each sub-region model:
intensity units which produce a value indicative of an average over the sub-region of a centre-surround intensity difference;
colour units which produce a value indicative of an average over the sub-region of a centre surround colour difference; and/or
direction-specific units which produce a value using filters which, for each of said direction-specific units, perform a filtering operation in a corresponding direction.

13. A method according to claim 12 in which there are a plurality of the intensity units for each sub-region, each intensity unit being modelled by forming an average over the sub-region of the magnitude of a difference I(c,s) between a first intensity function and a second intensity function, each of the intensity functions being defined over the image, and the second intensity function being obtained by sampling the first intensity function and interpolating values between the samples.

14. A method according to claim 12 in which the derivation of the numerical measures modelling the intensity units comprises:
convolving dyadic pyramids with the intensity channel of a colour image, to generate a plurality of intensity functions I(c), the intensity functions being labelled by an integer index c which takes values greater than 2 and represents of a corresponding scale in the image.

15. A method according to claim 12 in which there are a plurality of said numerical measures modelling colour units and obtained by:
using dyadic Gaussian pyramids and a colour function obtained from the image, to generate modified colour functions on a plurality of respective distance scales,
obtaining each numerical measure by forming an average over the sub-region of the magnitude of a difference between a first said modified colour function and a second said modified colour function, the first and second modified colour functions having different respective said distance scales.

16. A method according to claim 12 in which said numerical measures modelling direction-specific units are derived by for each of a plurality of directions:
filtering said image using a plurality of filters which each perform a filtering operation in that direction, the plurality of filters having different respective distance scales; and
pooling results of said filtering obtained using pairs of said filters.

17. A method according to claim 16 in which said filters are Gabor filters, the distance scale for each filter being a distance parameter used to define the corresponding filter using a Gabor mother function.

18. A method for treating an eye, the method including:
deciding whether an eye exhibits peripapillary atrophy (PPA), by
(i) deriving a region-of-interest within an image of the eve, the image being defined by intensity values at a set of pixels;
(ii) using the region-of-interest to derive a plurality of numerical measures (BIF), by subjecting the region to respective processing operations which mimic corresponding cortical visual processing operations; and
(iii) using said numerical measures to decide whether the eye exhibits peripapillary atrophy;
and in the case that the decision is positive treating the eye for a medical condition associated with PPA.

19. A computer system having a processor and a tangible data storage device, the data storage device storing non-transitory program instructions for performance by the processor to cause the processor to determine based on an image of an eye whether the eye exhibits peripapillary atrophy (PPA), the method comprising:
(i) deriving a region-of-interest within an image of the eye, the image being defined by intensity values at a set of pixels;
(ii) using the region-of-interest to derive a plurality of numerical measures (BIF), by subjecting the region to respective processing operations which mimic corresponding cortical visual processing operations; and
(iii) using said numerical measures to decide whether the eye exhibits peripapillary atrophy.
